(19) United States
(12) Patent Application Publication
(10) Pub. No.: US 2023/0002819 A1
(43) Pub. Date: Jan. 5, 2023

(54) Methods for Biomolecular Sensing and Detection

(71) Applicant: Universal Sequencing Technology Corporation, Canton, MA (US)

(72) Inventors: Peiming Zhang, Gilbert, AZ (US); Ming Lei, Sharon, MA (US); Kisup Chung, Weston, MA (US)

(21) Appl. No.: 17/636,612

(22) PCT Filed: Aug. 24, 2020
(86) PCT No.: PCT/US2020/047676

§ 371 Date: Feb. 18, 2022

Related U.S. Application Data
(60) Provisional application No. 62/890,251, filed on Aug. 22, 2019.

(51) Int. Cl. C12Q 1/6869; G01N 27/414; G01N 27/327

(52) U.S. Cl. CPC C12Q 1/6869; G01N 27/4145; G01N 27/4146; G01N 27/3276; G01N 27/3278

(57) ABSTRACT

The present invention relates to methods for creating conductive nanojunctions using metalized or conductive polymer joined to a DNA nanowire in a nanodevice for chemosensing and biosensing.

### CROSS-REFERENCE TO RELATED APPLICATIONS

This application claims priority to U.S. Provisional Application Ser. No. 62/890,251 filed Aug. 22, 2019, the entire disclosures of which are hereby incorporated herein by reference.

### FIELD

The present invention is related to systems, devices and methods for sensing biomolecules and biochemical reactions, including but not limited to the identification and/or sequencing of DNAs, RNAs, proteins, polypeptides, oligonucleotides, polysaccharides, and their analogies, etc., either natural, synthesized, or modified. More specifically, this disclosure includes embodiments in which patterned conductive nanojunctions are created in nanogaps using DNA as a scaffold or a template.

### BACKGROUND

An electronic nanodevice has great potential for biosensing in the point of care at a low cost. While a 7 nm resolution has achieved by Interference lithography,¹ the traditional top-down semiconductor fabrication process used in industry is approaching its limits. On the other hand, DNA is one of the most promising and suitable materials for integrating a biological operating system to the nanoelectronics with the angstrom precision. First, DNA can be programmed to form predictable nanometer-sized structures in both two and three dimensions by self-assembly, such as 2D DNA arrays, DNA-truncated octahedrons, DNA origamis, and 3D DNA.² Plus, sophisticated nucleic acid chemistry allows us to tune and modify DNA as well as create new DNA based materials. Thus, DNA has become a choice for the “bottom-up” construction of nanomachines.

DNA molecules could conduct electrons through overlapping 7c-orbitals of adjacent base pairs longitudinally. It has been observed that long native DNA wires are not conductive when deposited on a hard substrate,^{3,4} and short DNA molecules allow charge transport through them (<15 base pairs).⁵ In general, an AT sequence is less conductive than its GC counterpart in DNA.⁶ The AT base pair is considered a tunneling barrier, and the GC base pair a hopping site for the charge transfer. In aqueous solution, the conductance (G) of poly(CG)ₙ DNA duplexes decreases with their lengths (L).⁸ Although a poly(CG)₄ has a conductance about 100 nS (FIG. 2, a), an estimate for the poly(CG)ₙ to have a measurable conductance is about n=10 with the bias under 1V (FIG. 2, b). A prior study shows that a (polyG-polyC)₃₀ duplex only has a sub 1 nA conductance at room temperature and 50% air humidity under a 2 V bias (˜0.5 nS), (FIG. 1).⁷ Thus, DNA does not have sufficient conductivity over a range of necessary length scales for the nanoelectronics.

One way to improve the conductivity of a DNA nanostructure is to add conducting materials into DNA. For electronic interconnects, the nanostructure is better to have ohmic conductivity. Metallization of DNA is an effective way to create conductive nanowire. Metals, such as platinum (Pt), gold (Au), silver (Ag), copper (Cu), palladium (Pd), and rhodium (Rd), etc., have been plated on DNA to form metalized nanowires.⁹ In general, these DNA templated nanowires have their diameters greater than 10 nm for good conductivity. Braun and coworkers invented a molecular lithography technology for patterning on DNA substrates,¹⁰ where an insulating gap between two gold nanowires was created on a DNA substrate.

This invention provides means and methods to increase electrical conductivity of DNA nanojunctions by coating a thin layer of metal nanoparticles or conductive polymer monomers or conjugating a conductive polymer, such as polyaniline, or a combination of both along the DNA helices attached to a nanogap.

Pre-Remarks: Although this invention uses DNA duplex as a template or substrate or scaffold to make conductive nanowires or form nanojunctions, we do not exclude the use of other materials for the same purpose, such as an RNA duplex, a polypeptide chain, a polysaccharide chain, or similar biopolymers or the combination of them, including the combination with DNA, either natural or unnatural. The principles or methods of this invention apply to any other biopolymer suitable to be used as a nanowire/nanojunction building material.

### BRIEF DESCRIPTION OF THE DRAWINGS

FIG. 1: Conductivity of DNA composed of GC base pairs on the solid substrate, measured by a nanogap DNA junction. The DNA molecule (30 base pairs, double-stranded poly(G)-poly(C)) is 10.4 nm long, and the nanoelectrode gap is 8 nm wide. Current±voltage curves measured on a DNA molecule trapped between two metal nanoelectrodes at room temperature and 50% air humidity. Subsequent I-V curves show similar behavior but with a variation of the width of the voltage gap.

FIG. 2: Conductivity of DNA composed of GC base pairs in solution, measured by STM break junctions. (a) conductance histogram of a poly(GC)₈ DNA duplex; (b) Conductance of (GC)ₙ vs. 1/length.

FIG. 3: illustrates a top-down process of fabricating nanogaps.

FIG. 4: illustrates a bottom-up molecular lithograph process of fabricating nanojunctions to which a sensing molecule is attached, where an enzyme is shown as an example of the sensing molecule.

FIG. 5: showing tapered electrode end surface at the nanogap and a gate electrode underneath the nanogap.

FIG. 6: showing a vertical nanogap formed by electrodes in different planes separated by an insulation layer.

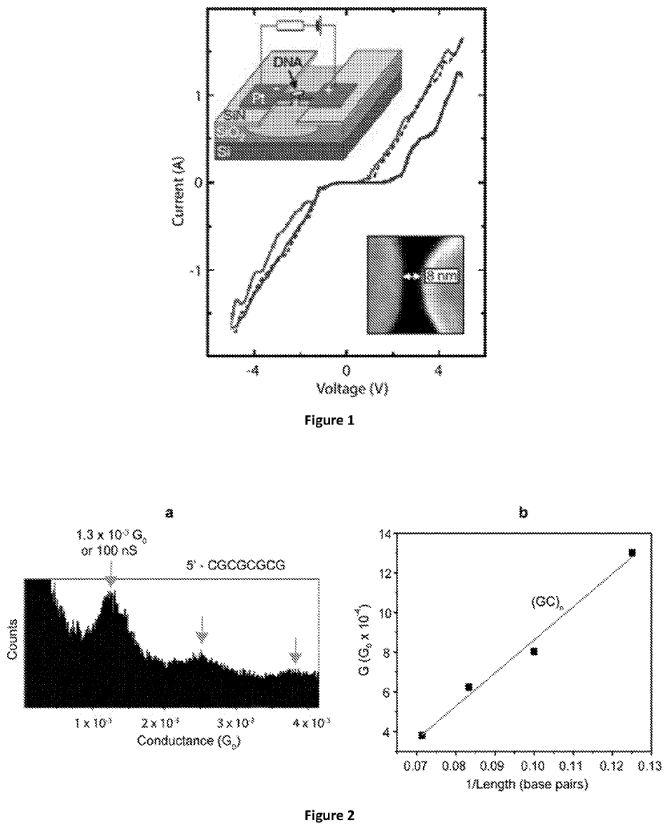

### SUMMARY OF THE INVENTION

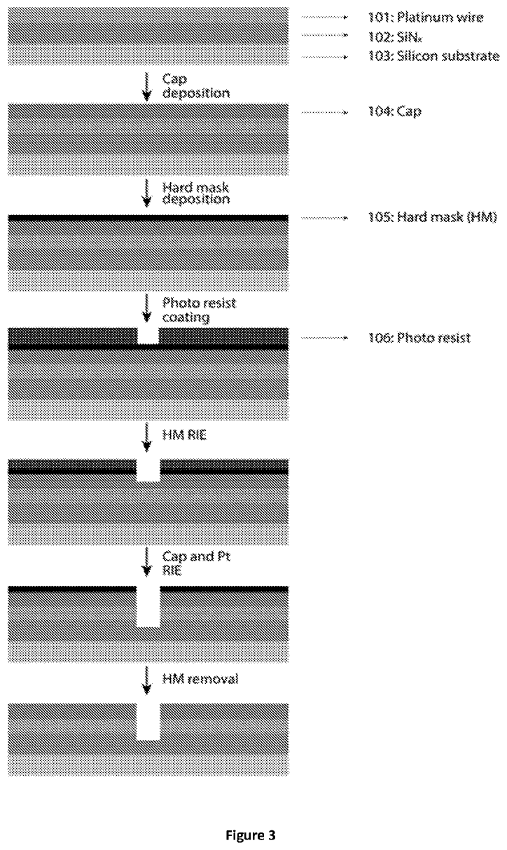

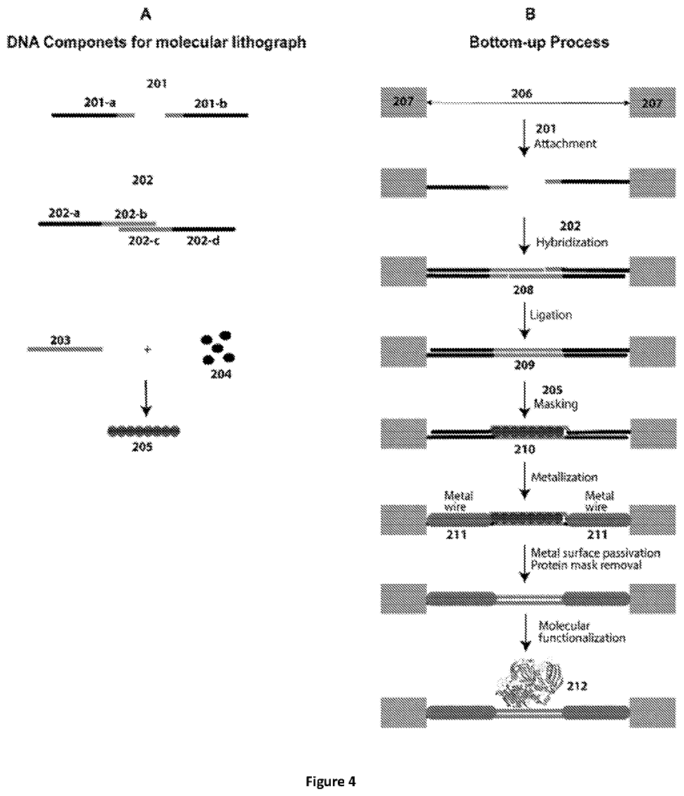

This invention provides methods to assemble a nanogap device for sensing biomolecules and biochemical reactions. FIG. 3 shows a scheme of fabricating nanogaps composed of two nanoelectrodes using standard semiconductor fabrication technologies in a top-down approach. Thus, the nanogaps can be fabricated with high yields and low cost. To bridge the nanogap with a molecular wire, a bottom-up molecular lithograph is applied to complete the whole process of the nanodevice assembly, as shown in FIG. 4.

In some embodiments, the nanogap comprises two electrodes, the distance between which is in a range of 3 nm to 1000 nm, preferably 5 nm to 100 nm, and most preferably 5 nm to 30 nm. The end surfaces of the electrodes are substantially rectangular with a width in the range of 3 nm to 1 um, preferably 5 nm to 30 nm, and a height in the range of 3 nm to 100 nm, preferably 5 nm to 30 nm. The said electrodes comprise noble metals, for example, platinum (Pt), gold (Au), silver (Ag), palladium (Pd), rhodium (Rd), ruthenium (Ru), osmium (Os), iridium (Ir), or other metals, such as copper (Cu), rhenium (Re), titanium (Ti), Niobium (Nb), Tantalum (Ta), and their derivatives, such as TiN, and TaN, etc.

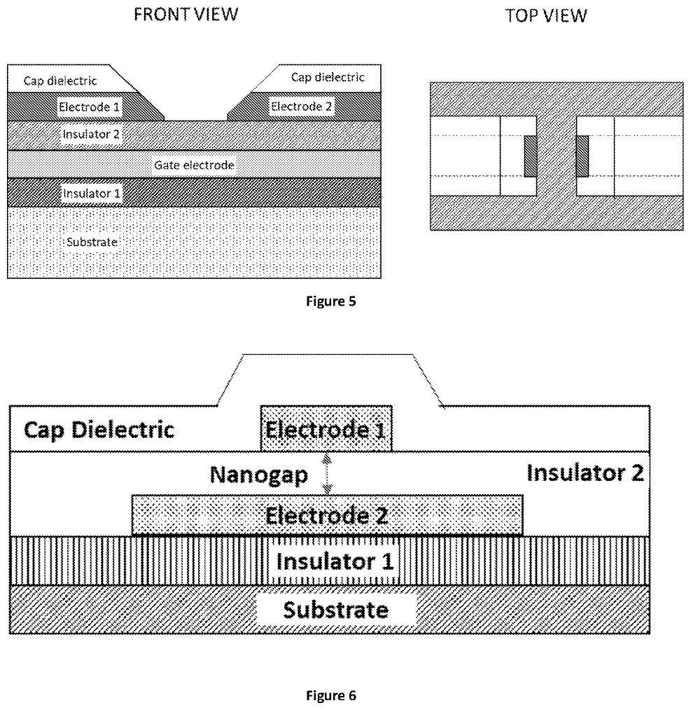

In some embodiments, the nanogap is formed by two electrodes which are in different planes separated by an insulation layer, see FIG. 6 (ref. U.S. 62/994,712). The thickness of the insulation layer is in the range of 2 nm to 1000 nm, preferably 5 nm to 30 nm. The insulation material is selected from, but not limited to, the group consisting of SiNx, SiOx, HfOx, Al₂O₃, other metal oxides, and any dielectrics used in the semiconductor industry.

In some embodiments, a nanojunction is formed by bridging the nanogap with a nanowire, and then a sensing molecule is attached at a predefined location. The said nanowire comprises a semiconductive DNA duplex segment flanked by two metalized or conductive polymer conjugated nanowire segments. A sensing molecule is attached to the DNA duplex in the middle. The attached sensing molecule and the semiconductive DNA duplex constitute a force effect transistor, referred as to “FET”. The sensing molecule changes its conformation when interacting with its receptors or substrates. This will exert a force on DNA and disturb its base stacking, resulting in fluctuation of electrical current flowing through the nanowire. The current signal, representing responses to the molecular events, is then recorded and the molecular interactions or reactions are deduced. For example, when the sensing molecule is a DNA polymerase, it can monitor the process of polymerase incorporating nucleotides into a DNA primer by recording the electric signals. When an antibody is used as the sensing molecule, an antigen can be detected utilizing this nanojunction device, or vice versa. Similarly, a receptor is used as a sensing molecule, and its ligands in a sample can be determined, or vice versa.

### DETAILED DESCRIPTION OF THE INVENTION

FIG. 3 delineates the process of fabricating a nanogap comprising two electrodes separated by sub-20 nm distance using the top-down semiconductor fabrication approach. A platinum wire (101) is fabricated on a silicon substrate (103) coated with a silicon nitride insulation layer (102) by EBL, and then a dielectric CAP layer (104) deposited, followed by a hard mask (HM) layer (105). A nanogap is fabricated by EBL patterning on a photoresist (106), followed by HM RIE, CAP, Pt RIE, and HM removal.

FIG. 4 delineates a bottom-up approach for the assembling of a nanojunction with the said nanogap. First, DNA anchors (201) are attached to the sidewalls of two electrodes (207) that define a nanogap (206). Then, a DNA wire (202) is hybridized to the DNA anchors to bridge the nanogap to form a nanojunction composed of double-stranded DNA having two nicks (208). The nicks are closed by a ligation reaction, resulting in a semiconductive DNA duplex segment (209). Then, a protein filament (205) is added to mask the middle section (210), followed by the deposition of metal particles on the flank segments to generate conductive wires (211) at the two ends of the semiconductive segment. After removing the protein filament, the semiconductive DNA duplex segment is exposed for attaching the sensing molecules, such as, an enzyme, a polymerase, or an antibody (212). This process provides a method to create a biomolecular sensing nanodevice.

In one embodiment, the said DNA anchor 201 is a set of short oligonucleotides 201-a and 201-b with their sequence matching to the DNA wire 202 at different ends. The probe (anchor) oligo 201-a matches to both 202-a and a part of 202-b. In the same way, 201-b matches to both 202-c and a part of 202-d. The sequences for the probe oligos 201-a and 201-b are either the same or different. When each of them is respectively attached to those individual electrodes constituting a nanogap, they capture the DNA wire 202 to form a duplex containing nicks. After a ligating reaction, a perfect duplex forms, the middle of which comprises a semiconductive segment (209) and the rest of which can be either conductive, semiconductive or non-conductive. Due to the complementary requirement for the hybridization (capture) of 202 to 201, it requires the nanogap size to match the length of the assembled molecular wire 209.

In another embodiment, the DNA anchor 201 is hybridized to the DNA wire 202 first, forming a DNA duplex with nicks to be filled by ligation, and then attaching to the two electrodes to form the nanojunction. In order to increase the success rate of junction formation, the ends of the electrodes can be tapered as a reverse trapezoidal geometry, see illustration in FIG. 5 (ref. U.S. 62/833,870), to facilitate the landing and attaching of the DNA duplex.

In some embodiment, the DNA anchor contains a functional group configured for attachment to the electrodes. The functional group includes but not limited to (a) a thiol on a sugar ring of a nucleoside; (b) a thiol and a selenol on a nucleobase of a nucleoside; (c) an aliphatic amine on a nucleoside; (d) a catechol on a nucleoside; (e) RXH and RXXR, where R is an aliphatic or aromatic group; X is chalcogens preferring to S and Se; and (f) Base chalcogenated nucleosides. For detailed descriptions of these functional groups it refers to U.S. 62/812,736.

In some embodiments, a third electrode, the gate electrode, is introduced, see FIG. 5 (ref. U.S. 62/833,870), and a reference voltage is applied to adjust the conductivity of the nanowire. The gate electrode is separated from the first and the second electrodes by a second insulation layer.

In another embodiment, the DNA anchor 201 and the DNA wire 202 are hybridized and ligated before attaching to the electrodes or simply replaced by a pre-assembled DNA duplex with the same sequence of DNA duplex 209. The pre-assembled DNA duplex is attached to the two electrodes at the nanogap, forming a nanojunction, followed by the protein filament attachment (masking) to the middle section and the metalization of the side sections as well as the final sensing molecule attachment (see FIG. 4). Again, it may require tapered electrode ends for better attachment or a gap smaller than or equal to the length of the pre-assembled DNA duplex.

In some embodiment, the nanowire comprises a semiconductive DNA duplex segment flanked by a metalized or conjugated conductive polymer segment at only one end. The sensing molecule is attached to pre-defined locations on the semiconductive DNA duplex segment.

In some embodiments, a pre-assembled DNA nanostructure constructed using methods disclosed in the previous provisional applications, U.S. 62/794,096 and U.S. 62/833,870, is used in place of DNA duplex 209, attaching to the electrodes directly to form nanojunction with the middle part compatible with the DNA/protein filament 205 for masking, followed by the remaining steps in FIG. 4 to complete the biosensing nanodevice construction. Examples of these pre-assembled DNA nanostructures including but not limited to a single DNA or RNA duplex, a DNA/RNA mixed duplex, a double DNA duplex, a triple DNA duplex, a DNA origami structure, a DNA nanostructure, a peptide nanostructure, a PNA (peptide nucleic acid) nanostructure, a mixed DNA/PNA nanostructure or any DNA or RNA or PNA nanostructure with a middle section compatible with the protein filament for masking, either natural, unnatural, modified or synthesized or the combination thereof, wherein the middle section can a DNA duplex with high GC content (about 51% to 95%) or with modified DNA bases that make the DNA duplex semiconductive or conductive.

In some embodiments, the DNA wire 202 and DNA anchor 201 are complementary to the full length so that the resulting DNA duplex 209 is double stranded in its full length. While in some other embodiments, the DNA wire 202 is shorter than the nanogap size, not fully complementary DNA anchor 201, so forming a DNA duplex (209) with single-stranded oligonucleotide flanked at both ends. Either the DNA duplex 209 is all double-stranded or partial double stranded with end segments single-stranded, the remaining process for constructing the biosensing nanodevice is the same as that in FIG. 4. Either single or double stranded, the metalization process of the side (end) segments is similar.

In some embodiments, the mid-section of the DNA duplex 209 carries functional groups at pre-defined nucleotides (locations), which can carry out chemical reactions to connect other entities, such as a sensing molecule, to the wire.

In some embodiments, the end segments of the DNA duplex (209) comprise phosphorothioate oligonucleotides with a structure as illustrated below:

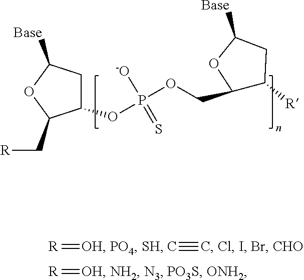

Where n=3 to 100; R and R′ can be a variety of functional groups as listed above, but not limited to them. The phosphate/phosphorothioate (PO/PS) chimeric oligodeoxyribonucleotides can be synthesized in an automated DNA synthesizer.¹¹ The said ligation in FIG. 4 could be an autonomous chemical reaction between R and R′ or an enzyme-catalyzed process.

In some embodiments, the said filament (205) comprises a single-stranded DNA (203) with its sequence complementary or similar (with at least about 50% sequence homology to the nucleic acid duplex segment) to the sequence of the semiconductive middle segment of the DNA wire 202 (or the mid-section of the DNA duplex 209), and a protein, such as a RecA protein (204) that can be polymerized on the single DNA strand.¹² The filament can specifically bind to a homologous double-stranded DNA, and used as a mask for molecular lithography.¹⁰ In some embodiments, the said filament 205 binds to the semiconductive section of the DNA duplex (209) as a mask (210) for the metal deposition (plating) on the end segments of the DNA duplex.

In one embodiment, as an example of DNA metalization, the said metal nanowire 211 is prepared first by seeding ˜1.0 nm silver nanoparticles on the phosphorothioate via the metal thiol covalent bond, followed by washing with water to remove the excess silver nanoparticles. Then, a solution of KSCN (0.6 M) mixed with KAuCl₄ (0.06 M) in a 1:1 ratio is added to the nanojunction area, followed by the addition of hydroquinone (25 mM) in the same volume with the gold plating solution. The nanojunction is incubated in the solution for 60 seconds. Then, the solution is flashed out, and the nanojunction is rinsed with water. As a result, the gold nanowire is formed at the two side segments of the DNA junction. Sequentially, the gold wire is passivated by forming a hydrophilic monolayer, for example, an oligo(ethylene glycol) monolayer on the surface to prevent the nonspecific adsorption. The filament mask is removed by protein digestion using Proteinase K to expose the semiconductive DNA segment.

In some embodiments, the seeding nanoparticles are gold in place of silver. A noble metal, either the same or different from the first electrode and/or the second electrode, is deposited on the DNA nanojunction by nanoparticle directed electroless plating. The noble metal includes but not limited to Au, Ag, Pd, Pt, Rd, etc.

In some embodiments, the plating process is carried out by an electrochemical process to specifically deposit different metals in the defined locations.

In some embodiments, the metal is deposited on the DNA duplex with well-defined metal nanoparticle seeding without employing the DNA/protein filament mask 205.

In some embodiments, the phosphorothioate groups in the DNA duplex 209 reacts with 4-Bromobutyraldehyde, resulting in aldehyde functionalized phosphorothioates, as shown below:

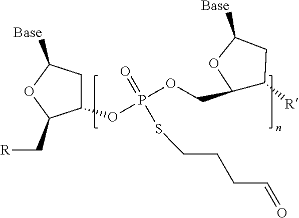

The aldehyde is a reducing agent for the seeding with a metal ion solution, for example, an AgNO₃ solution. Other aldehydes can also be used to functionalize the phosphorothioates, which have a structure, as shown below:

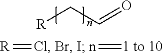

In some embodiments, the metalization of the end segments of the DNA duplex 209 can be replaced by co-joining a conductive polymer into the DNA segments. The DNA anchor (201) bears the monomers of conductive polymers (CP) attached to its nucleobases, or more generally, conductive polymer monomer coupled to the single-stranded end segments of the DNA duplex 209. The structures of the monomers are shown below, including, but not limited to:

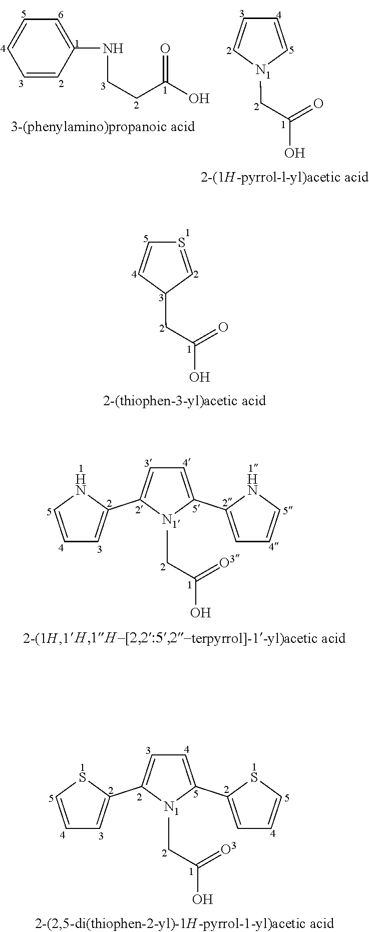

These monomers can be attached to the modified nucleosides with their nucleobases functionalized with the amine, as shown below:

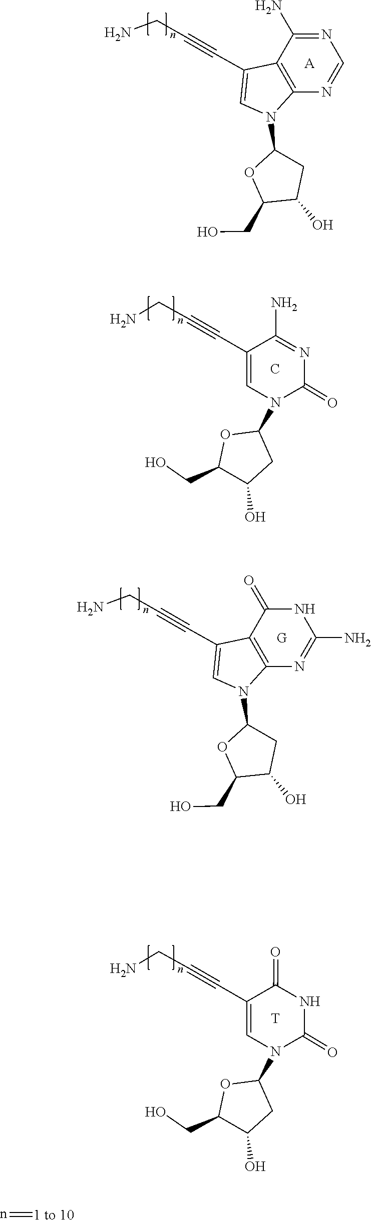

Thus, these functionalized nucleosides can be incorporated into DNA oligonucleotides by an automated DNA synthesizer. An example of synthesizing terpyrrole-uridine phosphoramidite (11) is described in the EXAMPLE section. The phosphoramidite (11) is incorporated into DNA, for example, with a sequence of CXA GXT AXC GXC by an automated DNA synthesizer, where X=uridine bearing a terpyrrole monomer. The DNA is used as an anchor for attaching to the electrodes. It hybridizes with the DNA wire to form a nanojuction in the nanogap, and they are ligated together. The protein filament mask is added to the semiconductive segment. Then, the terpyrrole monomers are polymerized by electrochemical oxidation in aqueous solution at neutral pH following a prior art approach.¹³ After removing the mask, the nanojunction is ready to be functionalized with sensing molecules. Alternatively, the terpyrrole monomers are conjoined and polymerized along the entire DNA duplex 209 without using the protein filament mask 205.

In some embodiments, the said DNA anchor bearing the CP monomers is prepared first by synthesizing a DNA oligonucleotide bearing amino-functionalized nucleosides, and then the CP monomers are coupled to the oligonucleotide by a reaction of activated carboxylate with the amine.

In some embodiments, the said conductive polymer in the DNA nanojunction is synthesized by either chemical or enzymatic oxidation, which has been demonstrated in prior arts.^{14, 15}

In some other embodiments, the conductive polymer is joined throughout the entire DNA duplex 209, not limited to the end segments, making the whole nanojunction comprising a conductive polymer joined to the DNA scaffold. Meanwhile, some functional groups, such as azide, thiol and its derivatives, are placed in the pre-defined locations along the DNA duplex for the attachment of the sensing molecule.

In some embodiments, conductive polymer monomers are conjugated to a DNA template or scaffold in an aqueous solution with or without a protein filament mask to form a conductive nanowire first and then the nanowire is attached to the first and the second electrodes to bridge the nanogap so to form a conductive nanojunction.

In other embodiments, first, conductive polymer monomers are deposited onto a DNA template or scaffold in an aqueous solution with or without a protein filament mask; second, the DNA template or nanowire is attached to the first and the second electrode to bridge the nanogap, and third, the conductive polymer monomers are oxidized to enhance the nanowire conductivity so a conductive nanojunction is formed.

In some embodiments, the nanowire or its underline DNA or polymer scaffold or template carries functional groups at its ends, such as azide, alkyne, or thiol and its derivatives, for the attachment to the first and the second electrodes.

In some embodiments, the DNA nanowire 209 is a duplex or a mixture of duplex with single stranded segments at the ends. In some embodiments, the DNA nanowire 209 is a triplex or a mixture of duplex, triplex, and single-stranded segments.

In some embodiment, a conductive polymer can be joined to the DNA nanowire at the end, forming a DNA-conductive polymer conjugated nanowire.

In some embodiment, the DNA scaffold underline the metalized DNA nanowire segment(s) can be replaced by any polymer that can be metalized and be joined to the DNA duplex nanowire segment, either the polymer is conductive, semiconductive or non-conductive, and either it is natural or unnatural.

In all the above, the conductive polymer is selected from, but not limited to, the group consisting of polypyrroles (PPY), polythiophenes (PT), polyanilines (PANI), poly(p-phenylene sulfide) (PPS), poly(acetylene)s (PAC), poly(p-phenylene vinylene) (PPV), poly(3,4-ethylenedioxythiophene) (PEDOT), poly(fluorene)s, polyphenylenes, polypyrenes, polyazulenes, polynaphthalenes, polycarbazoles, polyindoles, polyazepines, etc. The first three polymers, PPY, PT and PANI, are preferred due to relative easiness for synthesis.

In some embodiments, the sensing molecule is selected from, but not limited to, the group consisting of nucleic acid probes, molecular tweezers, enzymes, receptors, ligands, antigens and antibodies, either native, mutated, expressed, or synthesized, and a combination thereof.

In some embodiments, the sensing molecule is an enzyme, including but not limited to DNA polymerase, RNA polymerase, DNA helicase, DNA ligase, DNA exonuclease, reverse transcriptase, RNA primase, ribosome, sucrase, lactase, etc., either natural, mutated or synthesized.

In some embodiments, the sensing molecule is DNA polymerase, including but not limited to, any polymerase from polymerase families, A, B, C, D, X, Y, and RT. For example, those in Family A include T7 DNA polymerase and Bacillus stearothermophilus Pol I; those in Family B include T4 DNA polymerase, Phi29 DNA polymerase, and RB69; those in Family C includes the E. coli DNA Polymerase III. The RT (reverse transcriptase) family of DNA polymerases includes, for example, retrovirus reverse transcriptases and eukaryotic telomerases.

In some other embodiments, the sensing molecule is RNA polymerase, including but not limited to, viral RNA polymerases such as T7 RNA polymerase; Eukaryotic RNA polymerases such as RNA polymerase I, RNA polymerase II, RNA polymerase III, RNA polymerase IV, and RNA polymerase V; and Archaea RNA

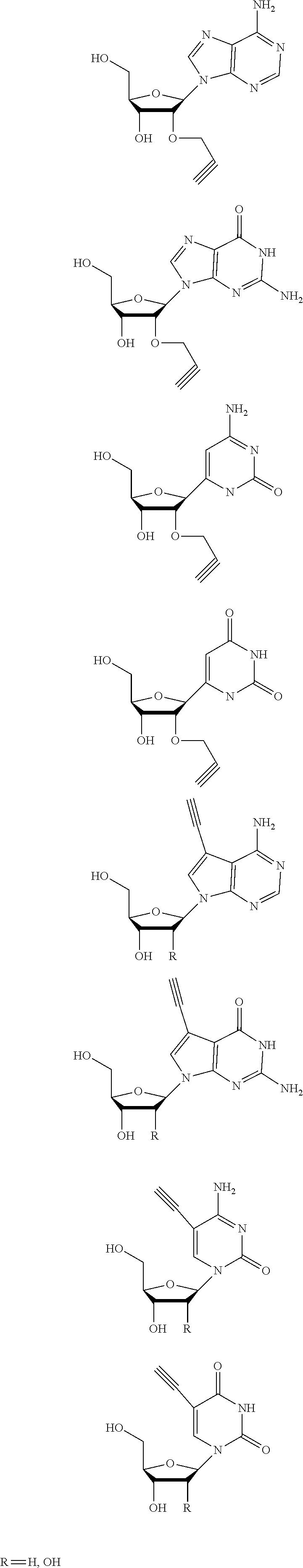

polymerase.

In some embodiments, click reactions are used to attach sensing molecules onto nanojunctions. As an example, nucleosides containing acetylene are incorporated into the conductive DNA segments for attaching sensing molecules functionalized with azide, following methods disclosed in our PCT filing (WO 2020/150695). Their structures are shown below

In some embodiments, a plurality of nanogap devices, each having all the features of a single nanogap device with attached nanowire and sensing molecule, can be fabricated in an array format with the number of nanogap devices from 10 to 10⁹ on a nanochip, a solid surface or in a well, preferably 10³ to 10⁷ or more preferably 10⁴ to 10⁶ based on the throughput requirement of the biopolymer sensing or sequencing. All of the nanogap devices in the said array is configured with one type of sensing molecule or different types of sensing molecules.

### Example

Ethyl 2-(2,5-dibromo-1H-pyrrol-yl)acetate (4) is synthesized following the route shown below:

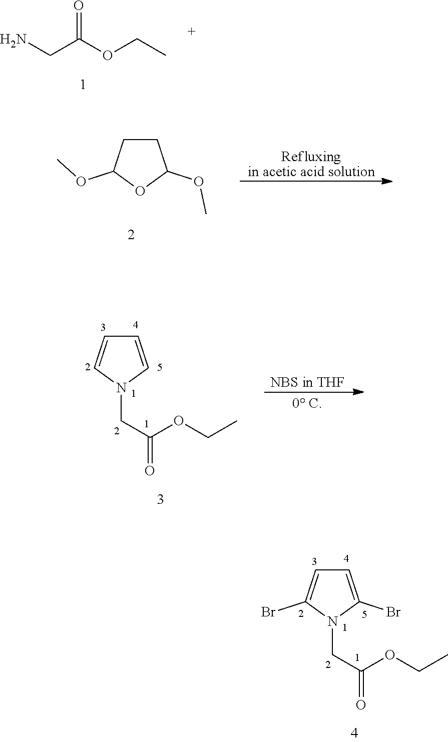

First, ethyl 2-(1H-pyrrol-yl)acetate (3) is synthesized following the procedure in a prior art (WO 2011/094823) with modifications. To a refluxing solution of ethyl glycinate (1, 1.0 equivalent) and sodium acetate (1.7 equivalents) in an appropriate solvent, such as a co-solvent of water/acetic acid (1:2), 2,5-dimethoxytetrahydrofuran (2, 1.0 equivalent) is added. The solution is refluxed for ˜4 hours, diluted with water, neutralized with a saturated aqueous solution of NaHCO₃, and extracted with CH₂Cl₂. The organic phase is dried over MgSO₄, filtrated, and concentrated by rotary evaporation. The residue is separated by flash chromatography on a silica column and the desired compound 3 obtained in a yield of >50%. In turn, brominated pyrrole 4 is synthesized following a procedure reported in the literature.¹⁶ A solution of N-bromosuccinimide (NBS, 2.0 equiv.) in anhydrous DMF was added dropwise to a solution of compound 3 (1.0 equiv.) in anhydrous THF at 0° C. After the addition, the mixture is stirred for 30 min. The reaction is monitored by TLC until finished, stopped by the addition of water, and extracted with chloroform three times. The combined organic solution is washed with water, dried over MgSO₄, filtered, and evaporated to remove the solvent. The residue is separated by flash chromatography over a silica column, furnishing the desired product with a yield of >90%.

(1-((2-(trimethylsilyl)ethoxy)methyl)-1H-pyrrol-2-yl)boronic acid (6) is synthesized following a procedure reported in the literature.¹⁷

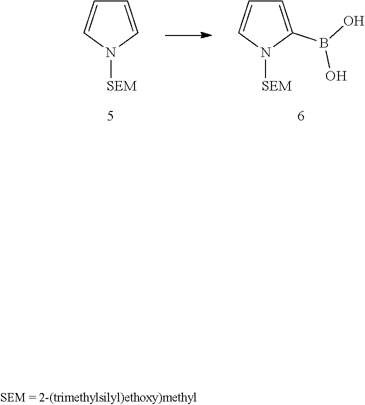 [SMILES: C[Si](C)(C)CCOCn1cccc1 ; C[Si](C)(C)CCOCn1cccc1B(O)O]

To a solution of 1-((2-(trimethylsilyl)ethoxy)methyl)-1H-pyrrole (5) in anhydrous THF at ˜78° C. under argon, a solution of lithium 2,2,6,6-tetramentylpiperidide (LiTMP) is added dropwise. The solution is stirred for four hours at ˜78° C., followed by the addition of triethylborate ((EtO)₃B) dropwise. The mixture is allowed to warm to room temperature for stirring an additional 12 h. The reaction mixture is quenched with saturated NH₄Cl solution and stirred for 40 min. The suspension was neutralized with saturated NaHCO₃ aqueous solution and stirred for 20 min. The solution is extracted three times with ether. The combined organic layer is dried over Na₂SO₄, and the solvent is removed by rotary evaporation. The residue is separated by flash chromatography on a silica column to obtained desired product 6.

2-(1,1″-bis((2-(trimethylsilyl)ethoxy)methyl)-1H,1′H,1″H-[2,2′:5′,2″-terpyrrol]-1′-yl)acetic acid (8) is synthesized following the route as shown below:

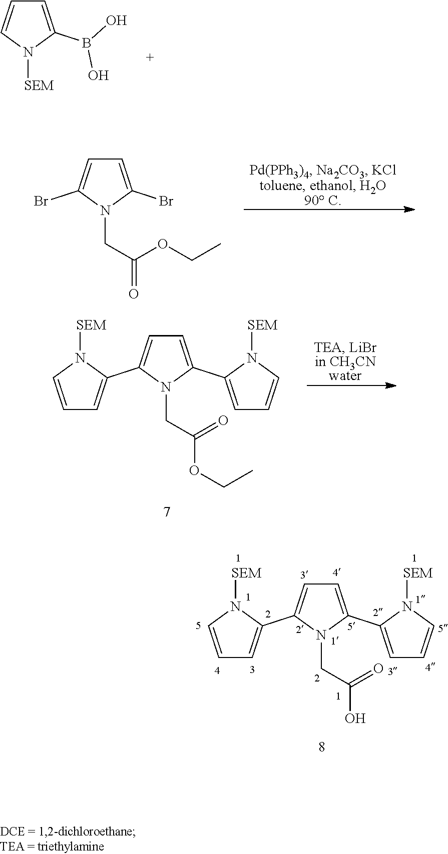

First, a terpyrrole ester is synthesized based on the method reported in the literature.¹⁸ The terpyrrole Pyrroleboronic acid 6 (2.3 eq.), tetrakis(triphenylphosphine)palladium(0) (10 mol %), sodium carbonate (8 eq.) and potassium chloride (3 eq.) are evacuated and flushed with argon twice. Then degassed toluene (20 mL), dibromopyrrole 4 (1 eq.), degassed ethanol and water are added. The mixture is heated for 18 h at 95° C., cooled and the solvents are removed by rotary evaporation. The residue is extracted with chloroform three times and the combined organic phase is washed with brine, dried over Na₂SO₄, and filtered. The solvent is removed by rotary evaporation. The residue is separated by silica gel gradient column chromatography to give the desired terpyrrole ester 7, which is converted to its corresponding carboxylic acid 8 following a mild hydrolysis procedure reported in the literature.¹⁹ The ester is dissolved in CH3CN (10 ml/g of ester) containing 2 vol % of water. Triethylamine (3 equiv.) is added, followed by the addition of LiBr (10 equiv.). The mixture is stirred vigorously at room temperature, and the product separated by silica gel gradient column chromatography.

2-(1,1″-bis((2-(trimethylsilyl)ethoxy)methyl)-1H,1 ‘H,1″H-[2,2’:5′,2″-terpyrrol]-1′-yl)-N-(3-(deoxyuridine-5-yl)prop-2-yn-1-yl)acetamide (10) is synthesized following a route as shown below:

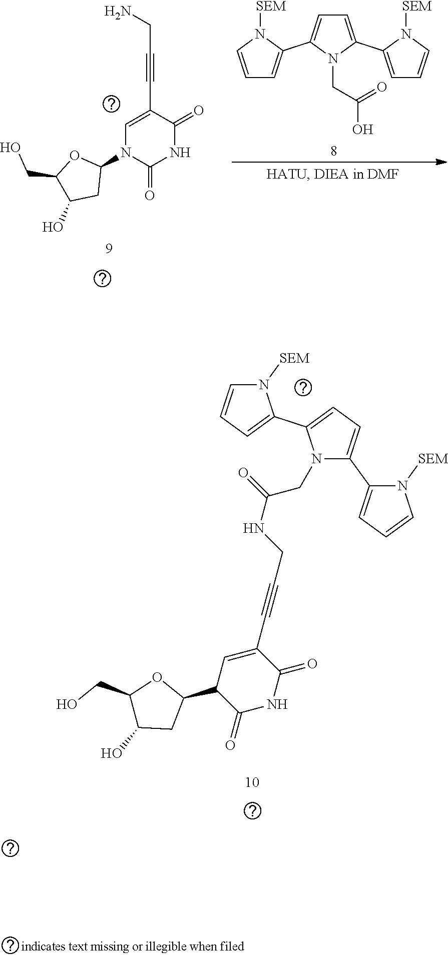 [SMILES: NCC#Cc1cn([C@H]2C[C@H](O)[C@@H](CO)O2)c(=O)[nH]c1=O ; C[Si](C)(C)CCOCn1cccc1-c1ccc(-c2cccn2COCC[Si](C)(C)C)n1CC(=O)O ; "HATU, DIEA in DMF" ; C[Si](C)(C)CCOCn1cccc1-c1ccc(-c2cccn2COCC[Si](C)(C)C)n1CC(=O)NCC#CC1=CC([C@H]2C[C@H](O)[C@@H](CO)O2)C(=O)NC1=O]

To a solution of 8 (200 mg, 1.0 equiv.) in DMF at 0° C., HATU (2.0 equiv.) and DIEA (3.0 equiv.) are added, followed by the addition of 5-(3-aminoprop-1-yn-1-yl)-deoxyuridine 9 (1.1 equiv.) The resulting mixture is stirred at RT for one hour, and then the reaction mixture is diluted with water and extracted with ethyl acetate three times. The organic layer was dried over sodium sulfate, filtered, and concentrated by rotary evaporation. The residue is separated by flash chromatography on a silica column, furnishing the desired product 10.

5′-O-dimethoxytrityl-2-(1,1″-bis((2-(trimethylsilyl)ethoxy)methyl)-1H,1 ‘H, 1″H-[2,2’:5′,2″-terpyrrol]-1′-yl)-N-(3-(deoxyuridine-5-yl)prop-2-yn-1-yl)acetamide-3′-O-(2-cannothyl-N,N-diisopropylphosphoramidite (11) is synthesized following the route shown below:

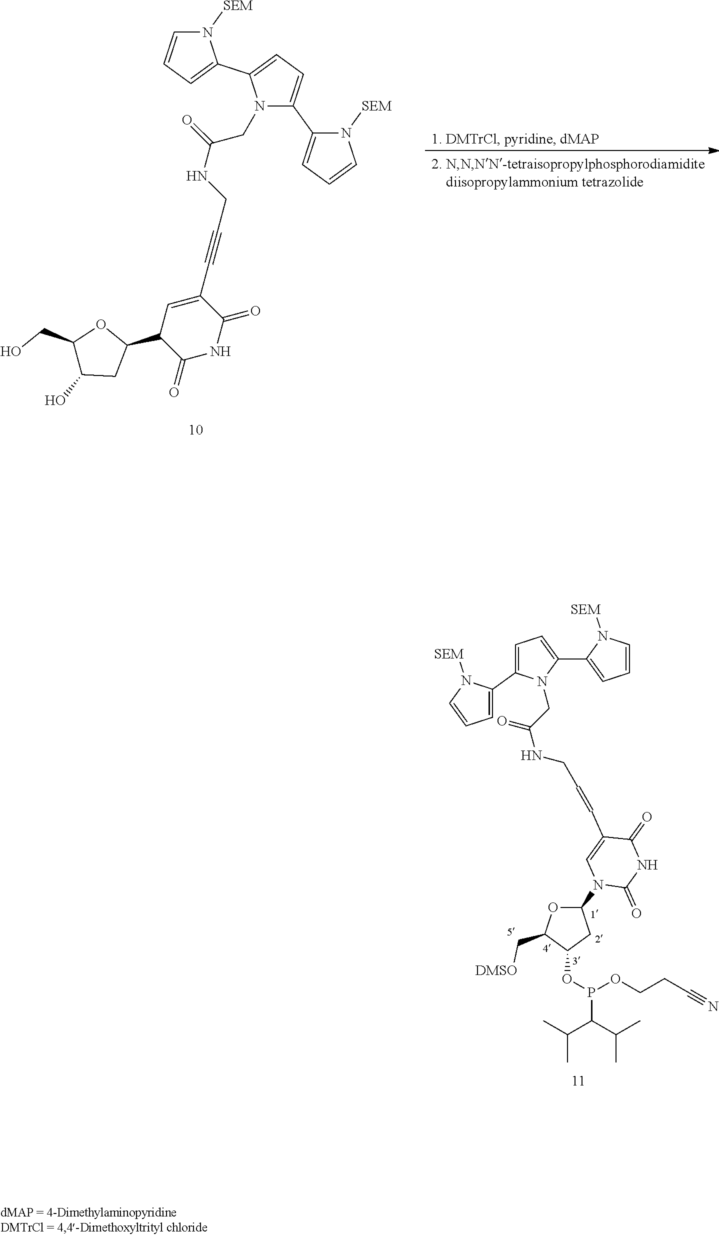

First, to a solution of modified deoxyuridine 10 (1.18 mmol) in pyridine (3 ml) is added 4,4′-dimethoxytrityl chloride (1.30 mmol). The mixture was stirred at room temperature for one hour. TLC analysis indicated the presence of a small amount of starting material. Additional 4,4′-dimethoxytrityl chloride is added to complete the reaction. The mixture is poured into water (50 ml) and extracted with methylene chloride (3×50 ml). The combined organic phase is washed with water and dried over anhydrous Na₂SO₄. The product is separated by flash chromatography on a silica gel column using a mixture of methylene chloride/methanol (95:5) as eluent. Then, the tritylated product (0.57 mmol) and diisopropylammonium tetrazolide (0.57 mmol) are dissolved in methylene chloride (6 ml). To the solution is added 2-cyanoethyl N,N,N′,N′-tetraisopropylphosphorodiamidite (0.66 mmol). The solution is gently swirled, allowed to stand under nitrogen at room temperature for 1.5 h, and then diluted with ethyl acetate, washed with water, and dried over anhydrous Na₂SO₄. The product was separated by silica gel chromatography on a chromatotron using a mixture of ethyl acetate/triethylamine (98:2) as eluent. Compound 11 is obtained as a foamed solid.

### GENERAL REMARKS

All publications, patents, patent applications, and other documents mentioned herein are incorporated by reference in their entirety. Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in art. The invention in its broader aspects is therefore not limited to the specific details, representative device, apparatus and method, and illustrative example shown and described. Accordingly, departures may be made from such details without departing from the spirit of the applicant's general inventive concept.

### REFERENCES

1 Mojarad, N., Gobrecht, J. & Ekinci, Y. Interference lithography at EUV and soft X-ray wavelengths: Principles, methods, and applications. Microelectronic Engineering 143, 55-63 (2015).
2 Xavier, P. L. & Chandrasekaran, A. R. DNA-based construction at the nanoscale: emerging trends and applications. Nanotechnology 29, 062001 (2018).
3 Storm, A. J., van Noort, J., de Vries, S. & Dekker, C. Insulating behavior for DNA molecules between nanoelectrodes at the 100 nm length scale. Applied Physics Letters 79, 3881-3883 (2001).
4 Zhang, Y., Austin, R. H., Kraeft, J., Cox, E. C. & Ong, N. P. Insulating Behavior of □-DNA on the Micron Scale. Physical Review Letters 89, 198102 (2002).
5 Guo, X., Gorodetsky, A. A., Hone, J., Barton, J. K. & Nuckolls, C. Conductivity of a single DNA duplex bridging a carbon nanotube gap. Nature Nanotechnology 3, 163, (2008)
6 Korol, R. & Segal, D. From Exhaustive Simulations to Key Principles in DNA Nanoelectronics. The Journal of Physical Chemistry C 122, 4206-4216 (2018).
7 Porath, D., Bezryadin, A., de Vries, S. & Dekker, C. Direct measurement of electrical transport through DNA molecules. Nature 403, 635-638 (2000).
8 Xu, B., Zhang, P., Li, X. & Tao, N. Direct Conductance Measurement of Single DNA Molecules in Aqueous Solution. Nano Lett. 4, 1105-1108 (2004).
9 Bayrak, T., Jagtap, N. S. & Erbe, A. Review of the Electrical Characterization of Metallic Nanowires on DNA Templates. Int. J. Mol. Sci. 19, 3019 (2018).
10 Keren, K. et al. Sequence-Specific Molecular Lithography on Single DNA Molecules. Science 297, 72-75 (2002).
11 Nukaga, Y., Oka, N. & Wada, T. Stereocontrolled Solid-Phase Synthesis of Phosphate/Phosphorothioate (PO/PS) Chimeric Oligodeoxyribonucleotides on an Automated Synthesizer Using an Oxazaphospholidine—Phosphoramidite Method. The Journal of Organic Chemistry 81, 2753-2762, (2016).
12 Cox, M. M. Motoring along with the bacterial RecA protein. Nature Reviews Molecular Cell Biology 8, 127-138 (2007).
13 Nhan, B. D. & Tuan, M. A. Electrochemical synthesis of polypyrrole for biosensor application. Int. J. Nanotechnology 10, 154-165 (2013).
14 Chen, W. et al. Development of Self-Organizing, Self-Directing Molecular Nanowires: Synthesis and Characterization of Conjoined DNA-2,5-Bis(2-thienyl)pyrrole Oligomers. Macromolecules 43, 4032-4040 (2010).
15 German, N., Popov, A., Ramanaviciene, A. & Ramanavicius, A. Enzymatic Formation of Polyaniline, Polypyrrole, and Polythiophene Nanoparticles with Embedded Glucose Oxidase. Nanomaterials 9, 806 (2019).
16 Faigl, F. et al. A novel and convenient method for the preparation of 5-(diphenylmethylene)-1H-pyrrol-2(5H)-ones; synthesis and mechanistic study. Tetrahedron 72, 5444-5455 (2016).
17 Kelly, T. A., Fuchs, V. U., Perry, C. W. & Snow, R. J. The efficient synthesis and simple resolution of a prolineboronate ester suitable for enzyme-inhibition studies. Tetrahedron 49, 1009-1016 (1993).
18 Brewster, J. T. et al. Gram-Scale Synthesis of a Bench-Stable 5,5″-Unsubstituted Terpyrrole. The Journal of Organic Chemistry 83, 9568-9570 (2018).
19 Mattsson, S., Dahlström, M. & Karlsson, S. A mild hydrolysis of esters mediated by lithium salts. Tetrahedron Letters 48, 2497-2499 (2007).

What is claimed:

1. A system for identification, characterization, or sequencing of a biopolymer comprising,
a. a substrate;
b. a nanogap formed by a first electrode and a second electrode placed next to each other on the substrate;
c. a nanowire that has a dimension configured to approximate the nanogap and configured to bridge the nanogap by attaching one end of the nanowire to the first electrode and another end of the nanowire to the second electrode, wherein the nanowire comprises a nucleic acid duplex segment flanked by at least a metalized polymer segment or at least a conductive polymer segment at the ends of the nucleic acid duplex segment;
d. a sensing molecule is configured to be attached to the nucleic acid duplex segment on the nanowire that is configured to interact or to perform a biochemical reaction with the biopolymer;
e. a bias voltage configured to be applied between the first electrode and the second electrode;
f. a device configured to record a current fluctuation through the nanowire caused by the activity of the sensing molecule; and
g. a software configured for data analysis that identifies or characterizes the biopolymer or a subunit of the biopolymer.

2. The system of claim 1 further comprises an insulation layer 1 between the substrate and the first and the second electrodes.

3. The system of claim 1 further comprises a dielectric cap layer on top of the electrodes.

4. The system of claim 1 further comprises
a. a gate electrode, separated from the first and the second electrodes by an insulation layer 2; and
b. a reference voltage configured to be applied to the gate electrode.

5. The system of claim 1, wherein the biopolymer is selected from the group consisting of a DNA, a RNA, an oligonucleotide, a protein, a polypeptide, a polysaccharide, an analog of any of the aforementioned biopolymers, either natural, modified or synthesized of any of the aforementioned biopolymers, and a combination thereof.

6. The system of claim 1, wherein the sensing molecule is selected from the group consisting of a nucleic acid probe, a molecular tweezer, an enzyme, a receptor, a ligand, an antigen and an antibody, either native, mutated, expressed, or synthesized, and a combination thereof.

7. The system of claim 6, wherein the enzyme is selected from the group consisting of a DNA polymerase, a RNA polymerase, a DNA helicase, a DNA ligase, a DNA exonuclease, a reverse transcriptase, a RNA primase, a ribosome, a sucrase, a lactase, either native, mutated, expressed, or synthesized of any of the aforementioned enzymes, and a combination thereof.

8. The system of claim 7, wherein the DNA polymerase or the enzyme is selected from the group consisting of an □29 DNA polymerase, a T4 DNA polymerase, a T7 DNA polymerase, a Taq polymerase, a RB69 polymerase, a DNA polymerase X, a DNA polymeraseY, a DNA Polymerase Pol I, a Pol II, a Pol III, a Pol IV and a Pol V, a Pol □ (alpha), a Pol □ (beta), a Pol □ (sigma), a Pol □ (lambda), a Pol □ (delta), a Pol □□□ epsilon), a Pol μ (mu), a Pol □ (iota), a Pol □ (kappa), a Pol □ (eta), a terminal deoxynucleotidyl transferase, a retrovirus reverse transcriptase, a telomerase, either native, mutated, expressed, or synthesized of any of the aforementioned enzymes, and a combination thereof.

9. The system of claim 7, wherein the RNA polymerase is selected from the group consisting of a T7 RNA polymerase, any viral RNA polymerase, a RNA polymerase I, a RNA polymerase II, a RNA polymerase III, a RNA polymerase IV, a RNA polymerase V, any eukaryotic RNA polymerase, any archaea RNA polymerase, either natural, modified, expressed, or synthesized, and a combination thereof.

10. The system of claim 1, wherein the sensing molecule configured to be attached to the nucleic acid duplex segment of the nanowire at a predefined location through a click reaction.

11. The system of claim 1, wherein the nanogap size or the distance between the two electrodes, is in the range of about 3 nm to about 1000 nm, preferably about 5 nm to about 30 nm.

12. The system of claim 1, wherein the end surfaces of the electrodes facing the nanogap are substantially rectangular with a width in the range of about 3 nm to about 1 um, preferably about 5 nm to about 30 nm, and a height in the range of about 3 nm to about 100 nm, preferably about 5 nm to about 30 nm.

13. The system of claim 1, wherein the nanogap has an approximate reverse trapezoidal shape with an opening wider at the top than the nanowire length and an opening narrower at the bottom than the nanowire length.

14. The system of claim 1, wherein the electrodes are made from a material selected from the group consisting of platinum (Pt), gold (Au), silver (Ag), palladium (Pd), rhodium (Rd), ruthenium (Ru), osmium (Os), and iridium (Ir), copper (Cu), rhenium (Re), titanium (Ti), Niobium (Nb), Tantalum (Ta) and their derviatives, such as TiN, and TaN, and a combination thereof.

15. The system of claim 1, wherein the first electrode and the second electrode are in different planes, overlapping each other and separated by the insulation layer, wherein the nanogap size is defined by the approximate thickness of the insulation layer.

16. The system of claim 1, wherein the nucleic acid duplex segment is configured to be compatible with a protein filament for polymer metalization or molecular lithograph masking.

17. The system of claim 16, wherein the protein filament comprises a single strand nucleic acid sequence complementary or a sequence with at least about 50% sequence homology to the nucleic acid duplex segment.

18. The system of claim 1, wherein the nucleic acid duplex segment comprises a modified nucleic base that enhances the nucleic acid duplex segment conductivity, wherein the modified nucleic base is either natural or unnatural.

19. The system of claim 1, wherein the nucleic acid duplex segment comprises a modified nucleic base with a functional group for the attachment of the sensing molecule, wherein the modified nucleic base is either natural or unnatural.

20. The system of claim 19, wherein the functional group is an azide or a thiol group.

21. The system of claim 1, wherein the metalized polymer segment is made by seeding and/or depositing a metal particle onto a polymer substrate, wherein the polymer substrate is a portion or an extension of the nucleic acid duplex segment or a polymer joined to the nucleic acid duplex segment, wherein the polymer is either conductive, semiconductive or non-conductive, or a combination thereof.

22. The system of claim 21, wherein the metal particle is selected from the group consisting of platinum (Pt), gold (Au), silver (Ag), palladium (Pd), rhodium (Rd), ruthenium (Ru), osmium (Os), and iridium (Ir), and a metal same as the first electrode and/or the second electrode, and a combination thereof.

23. The system of claim 21, wherein the seeding metal particle comprises either a gold nanoparticle or a silver nanoparticle.

24. The system of claim 21, wherein the metalized polymer segment is covered by a passivation monolayer.

25. The system of claim 21, wherein the polymer is selected from the group consisting of a DNA duplex, a RNA dulex, a DNA/RNA duplex, a partial DNA duplex, a partial RNA duplex, a single strand DNA, a single strand RNA, a DNA nanostructure, a peptide nanostructure, a PNA nanostructure, a portion of any of the above mentioned biopolymers, and a combination thereof, either natural, unnatural, modified or synthesized.

26. The system of claim 1, wherein the nucleic acid duplex segment is replaced by a biopolymer segment that is selected from the group consisting of a double DNA duplex, a triple DNA duplex, a DNA origami structure, a DNA nanostructure, a peptide nanostructure, a PNA nanostructure, a mixed DNA and PNA nanostructure, and a combination thereof, either natural, unnatural, modified, or synthesized, wherein the biopolymer segment is configured to have a functional group for the attachment of the sensing molecule and is compatible with a protein filament for polymer metalization or molecular lithography masking.

27. The system of claim 1, wherein the conductive polymer segment is made by coating a conductive polymer monomer onto a nucleic acid scaffold or substrate through an enzymatic, an electrochemical, or a chemical oxidation conjugation, or a combination thereof.

28. The system of claim 1, wherein the conductive polymer segment comprises a polymer selected from the group consisting of a polypyrrole (PPY), a polythiophene (PT), a polyaniline (PANI), a poly(p-phenylene sulfide) (PPS), a poly(acetylene) (PAC), a poly(p-phenylene vinylene) (PPV), a poly(3,4-ethylenedioxythiophene) (PEDOT), a poly(fluorene), a polyphenylene, a polypyrene, a polyazulene, a polynaphthalene, a polycarbazole, a polyindole, a polyazepine, and a combination thereof, either natural, unnatural, modified or synthesized.

29. The system of claim 1, wherein the conductive polymer is extended throughout the entire nanowire with the nucleic acid duplex segment co-joined at or near the middle of the nanowire for the sensing molecule attachment.

30. The system of claim 1, wherein a plurality of nanogap devices, each having all the features of a single nanogap device with attached nanowire and sensing molecule, configured to be fabricated in an array format.

31. The system of claim 30, wherein the number of nanogap devices is from about 10 to about 10⁹ on a nanochip, a solid surface or in a well.

32. The system of claim 30, wherein the number of nanogap devices is from about 10⁴ to about 10⁶.

33. A method for identification, characterization, or sequencing of a biopolymer comprising,
a. providing a substrate;
b. forming a nanogap by placing a first electrode and a second electrode next to each other on the substrate;
c. providing a nanowire that is configured to have a dimension comparable to the nanogap, wherein the nanowire comprises a nucleic acid duplex segment flanked by at least a polymer segment at its end, wherein the polymer segment is conductive and joined to the nucleic acid duplex segment;
d. attaching the nanowire to the first electrode at one end and to the second electrode at the other end;
e. attaching a sensing molecule to a predefinded location on the nucleic acid duplex segment, wherein the sensing molecule is configured to interact or perform a biochemical reaction with the biopolymer;
f. applying a bias voltage between the first electrode and the second electrode;
g. providing a device that is configured to record a current fluctuation through the nanowire caused by the activity of the sensing molecule; and
h. providing a software for data analysis that identifies or characterizes the biopolymer or a subunit of the biopolymer.

34. The method of claim 33 further comprises providing an insulation layer 1 between the substrate and the first and the second electrodes.

35. The method of claim 33 further comprises providing a dielectric cap layer on top of the electrodes.

36. The method of claim 33 further comprises
a. providing a gate electrode, separated from the first and the second electrodes by an insulation layer 2; and
b. applying a reference voltage to the gate electrode.

37. The method of claim 33, wherein the biopolymer is selected from the group consisting of a DNA, a RNA, an oligonucleotide, a protein, a polypeptides, a polysaccharide, an analog of any of the aforementioned biopolymers, either natural, modified or synthesized of any of the aforementioned biopolymers, and a combination thereof.

38. The method of claim 33, wherein the sensing molecule is selected from the group consisting of a nucleic acid probe, a molecular tweezer, an enzyme, a receptor, a ligand, an antigen and an antibody, either native, mutated, expressed, or synthesized, and a combination thereof.

39. The method of claim 38, wherein the enzyme is selected from the group consisting of a DNA polymerase, a RNA polymerase, a DNA helicase, a DNA ligase, a DNA exonuclease, a reverse transcriptase, a RNA primase, a ribosome, a sucrase, a lactase, either native, mutated, expressed, or synthesized of any of the aforementioned enzymes, and a combination thereof.

40. The method of claim 39, wherein the DNA polymerase or the enzyme is selected from the group consisting of a □29 DNA polymerase, a T4 DNA polymerase, a T7 DNA polymerase, a Taq polymerase, a RB69 polymerase, a DNA polymerase X, a DNA polymeraseY, a DNA Polymerase Pol I, a Pol II, a Pol III, a Pol IV, a Pol V, a Pol □ (alpha), a Pol □ (beta), a Pol □ (sigma), a Pol □ (lambda), a Pol □ (delta), a Pol □□□epsilon), a Pol m (mu), a Pol □ (iota), a Pol □ (kappa), a Pol □ (eta), a terminal deoxynucleotidyl transferase, a retrovirus reverse transcriptase, a telomerase, either native, mutated, expressed, or synthesized of any of the aforementioned enzymes, and a combination thereof.

41. The method of claim 39, wherein the RNA polymerase is selected from the group consisting of a T7 RNA polymerase, any viral RNA polymerase, a RNA polymerase I, a RNA polymerase II, a RNA polymerase III, a RNA polymerase IV, a RNA polymerase V, any eukaryotic RNA polymerase, any archaea RNA polymerase, either natural, modified, expressed, or synthesized, and a combination thereof.

42. The method of claim 33, wherein the sensing molecule is configured to attach to the DNA duplex segment of the nanowire at a predefined location through a click reaction.

43. The method of claim 33, wherein the nanogap size or the distance between the two electrodes, are configured to be in the range of about 3 nm to about 1000 nm, preferably about 5 nm to about 30 nm.

44. The method of claim 33, wherein the end surfaces of the electrodes facing the nanogap are substantially rectangular with a width in the range of about 3 nm to about 1 um, preferably about 5 nm to about 30 nm, and a height in the range of about 3 nm to about 100 nm, preferably about 5 nm to about 30 nm.

45. The method of claim 33, wherein the nanogap has an approximate reverse trapezoidal shape with an opening wider at the top than the nanowire length and an opening narrower at the bottom than the nanowire length.

46. The method of claim 33, wherein the electrodes are made from a material selected from the group consisting of platinum (Pt), gold (Au), silver (Ag), palladium (Pd), rhodium (Rd), ruthenium (Ru), osmium (Os), iridium (Ir), copper (Cu), rhenium (Re), titanium (Ti), Niobium (Nb), Tantalum (Ta) and their derviatives, such as TiN, and TaN, and the combination thereof.

47. The method of claim 33, wherein the nucleic acid duplex segment comprises a modified nucleic base that enhances the nucleic acid duplex segment conductivity.

48. The method of claim 33, wherein the nucleic acid duplex segment comprises a modified nucleic base with a functional group for the attachment of the sensing molecule.

49. The method of claim 33, wherein the functional group is an azide or a thiol group.

50. The method of claim 33, wherein the polymer segment is made by coating a conductive polymer monomer to a nucleic acid scaffold or substrate through an enzymatic, an electrochemical, or a chemical oxidation conjugation, or a combination thereof, wherein the nucleic acid scaffold is a single strand nucleic acid sequence, a double strand nucleic acid sequence, a partial single strand and partial double strand nucleic acid sequencing, or a continuous part of the middle nucleic acid duplex or a combination thereof.

51. The method of claim 50, wherein the coating of a conductive polymer monomer is made either before or after the nanowire is attached to the electrodes.

52. The method of claim 33, wherein the polymer segment comprises a polymer selected from the group consisting of a polypyrrole (PPY), a polythiophene (PT), a polyaniline (PANI), a poly(p-phenylene sulfide) (PPS), a poly(acetylene) (PAC), a poly(p-phenylene vinylene) (PPV), a poly(3,4-ethylenedioxythiophene) (PEDOT), a poly(fluorene), a polyphenylene, a polypyrene, a polyazulene, a polynaphthalene, a polycarbazole, a polyindole, a polyazepine, and a combination thereof, either natural, unnatural, modified or synthesized.

53. The method of claim 33, wherein the polymer segment is extended throughout the nanowire with the nucleic acid duplex co-joined at or near the middle of the nanowire for the sensing molecule attachment.

54. The method of claim 33 further comprises after step d and before step e:
1) providing a protein filament that is configured to be compatible with the nucleic acid duplex segment or a predefined portion on the nucleic acid duplex segment;
2) attaching the protein filament to the nucleic acid duplex segment as a mask for the metalization of the adjacent polymer segment;
3) metalizing the polymer segment using a molecular lithography approach with the polymer segment as the substrate or template; and
4) removing the protein filament from the nucleic acid duplex segment;
wherein the polymer segment is either conductive, semiconductive or non-conductive.

55. The method of claim 54, wherein the polymer segment is a continuous part or an extension of the nucleic acid duplex segment.

56. The method of claim 54, wherein the protein filament comprises a single strand nucleic acid sequence complementary or a sequence with at least about 50% sequence homology to the nucleic acid duplex segment.

57. The method of claim 54, wherein the metalization of the polymer segment is made by seeding or depositing a metal particle onto the polymer substrate.

58. The method of claim 57, wherein the metal particle is selected from the group consisting of platinum (Pt), gold (Au), silver (Ag), palladium (Pd), rhodium (Rd), ruthenium (Ru), osmium (Os), and iridium (Ir), and a metal same as the first electrode and/or the second electrode, and a combination thereof.

59. The method of claim 57, wherein the seeding metal particle comprises either gold nanoparticle or silver nanoparticle.

60. The method of claim 54, wherein the polymer is selected from the group consisting of a DNA duplex, a RNA dulex, a DNA/RNA duplex, a partial DNA duplex, a partial RNA duplex, or a single strand DNA, a single strand RNA, a DNA nanostructure, a peptide nanostructure, a PNA nanostructure, a portion of any of the above mentioned biopolymers, and a combination thereof, either natural, unnatural, modified or synthesized.

61. The method of claim 54, wherein the metalized polymer segment is covered by a passivation monolayer.

62. The method of claim 33, further comprises metalizing the polymer segment without masking the nucleic acid duplex segment by a properly designed metal particle seeding, wherein the polymer segment is either conductive, semiconductive or non-conductive.

63. The method of claim 33, wherein the nucleic acid duplex segment is replaced by a biopolymer segment that is selected from the group consisting of a mixed DNA/RNA duplex, a double DNA duplex, a triple DNA duplex, a DNA origami structure, a DNA nanostructure, a peptide nanostructure, a PNA nanostructure, a mixed DNA and PNA nanostructure, and a combination thereof, either natural, unnatural, modified, or synthesized, wherein the biopolymer segment comprises a functional group for the attachment of the sensing molecule and is configured to be compatible with a protein filament for polymer metalization or molecular lithography masking.
